(19) United States
(12) Patent Application Publication
(10) Pub. No.: US 2022/0087563 A1
(43) Pub. Date: Mar. 24, 2022

(54) WRAPPED AND FILLED RF COIL

(71) Applicant: Siemens Healthcare GmbH, Erlangen (DE)

(72) Inventors: Ying Lun Wang, Shenzhen (CN); Jun You, Shenzhen (CN)

(73) Assignee: Siemens Healthcare GmbH, Erlangen (DE)

(21) Appl. No.: 17/482,647

(22) Filed: Sep. 23, 2021

(30) Foreign Application Priority Data
Sep. 23, 2020 (CN) 202011006810.X

(51) Int. Cl. A61B 5/055; G01R 33/34; A61B 5/00

(52) U.S. Cl. CPC A61B 5/055; A61B 5/6831; A61B 5/6843; G01R 33/34

(57) ABSTRACT

A coil assembly, an MRI system, and a method of using the coil assembly. The coil assembly includes: a flexible coil configured to cover a tissue under test in a wound fashion; a pressure adjuster configured to be integrated with the flexible coil, for the purpose of applying uniform pressure to the entire flexible coil such that the flexible coil fits the covered tissue under test; a pressure controller configured to control the pressure applied to the flexible coil by the pressure adjuster; and a connection member, constructed to connect the pressure controller to the pressure adjuster.

### TECHNICAL FIELD

The present disclosure relates to the field of MRI, in particular to a magnetic resonance coil assembly capable of being wound and having an adjustable degree of fitting, an MRI apparatus, and a method of using the magnetic resonance coil assembly.

### BACKGROUND

Magnetic resonance imaging (MRI) is a technology in which the phenomenon of magnetic resonance is used to perform imaging. An MRI system generally comprises: a cavity-shaped superconducting magnet, encircling gradient coils within the superconducting magnet, a cavity-shaped body coil located within the gradient coils, an examination platform bed board on which a patient is placed, and a local coil for covering some part of the patient, such as a knee coil, shoulder coil, spine coil, hand/wrist coil, foot/ankle coil, body array coil and head/neck coil, etc.

In order to receive magnetic resonance signals more effectively and achieve higher image quality, those skilled in the art have long been working hard to find a better implementation scheme for local coils.

### SUMMARY

In view of the above, in aspects of the present disclosure, a magnetic resonance coil assembly and an MRI system are proposed in one aspect, and a method for using the magnetic resonance coil assembly is proposed in another aspect, for the purpose of improving magnetic resonance imaging quality.

The magnetic resonance coil assembly provided in aspects of the present disclosure comprises: a flexible coil, constructed to cover a tissue under test in a wound fashion; a pressure adjustment unit, constructed to be integrated with the flexible coil, for the purpose of applying uniform pressure to the entire flexible coil such that the flexible coil fits the covered tissue under test; a pressure control unit, constructed to control the pressure applied to the flexible coil by the pressure adjustment unit; and a connection member, constructed to connect the pressure control unit to the pressure adjustment unit.

In one aspect, the pressure adjustment unit is an airbag; the connection member is an inflation tube; the pressure control unit is a hollow latex ball, or an electronic controller with a gas pump and a pressure relief means.

In one aspect, the magnetic resonance coil assembly further comprises: a fixed support, used to accommodate the flexible coil and the tissue under test that is covered by the flexible coil in a wound fashion, and to fix and position the flexible coil and the tissue under test when the airbag expands under inflation to fill the fixed support.

In one aspect, the pressure adjustment unit is an elastic element; the connection member is a pulling element; the pressure control unit is a tension control element.

In one aspect, the flexible coil comprises: a flexible coil body, comprising multiple rows of flexible antenna units for receiving magnetic resonance signals; a flexible protective base layer, comprising two protective base layers arranged on two sides of the flexible coil body, and used to carry and protect the flexible coil body; and a nylon clasp, arranged at two ends of the flexible protective base layer, in order to lock the position of the flexible coil covering the tissue under test in a wound fashion.

In one aspect, the magnetic resonance coil assembly further comprises: an electronics box, a first electric cable, a second electric cable and a current plug, wherein the electronics box is constructed to accommodate a printed circuit board and electronic components; the first electric cable is constructed to electrically connect the electric cable plug to the electronics box; the second electric cable is constructed to electrically connect the electronics box to the flexible coil.

An MRI apparatus proposed in aspects of the present disclosure comprises: the magnetic resonance coil assembly as described in any one of the aspects above.

Proposed in aspects of the present disclosure is a method of using the magnetic resonance coil assembly as described in any one of the aspects above, the method comprising: covering the tissue under test with the flexible coil in the magnetic resonance coil assembly in a wound fashion; controlling the pressure adjustment unit in the magnetic resonance coil assembly to apply uniform pressure to the entire flexible coil by operating the pressure control unit in the magnetic resonance coil assembly, so that the flexible coil fits the covered tissue under test.

In one aspect, the pressure adjustment unit is the airbag, the connection member is the inflation tube, and the pressure control unit is the hollow latex ball; the step of controlling the pressure adjustment unit in the magnetic resonance coil assembly to apply uniform pressure to the entire flexible coil by operating the pressure control unit in the magnetic resonance coil assembly, so that the flexible coil fits the covered tissue under test, comprises: inflating the airbag by squeezing the hollow latex ball, the airbag applying uniform pressure to the entire flexible coil while expanding under inflation, so that the flexible coil fits the covered tissue under test.

In one aspect, the method of use further comprises: putting the flexible coil and the tissue under test into a fixed support; inflating the airbag by squeezing the hollow latex ball, until the airbag fills the fixed support.

It can be seen from the above solution that because, in aspects of the present disclosure, the flexible coil is provided with a pressure adjustment unit integrated with the flexible coil and capable of applying uniform pressure to the entire flexible coil 1, and is also provided with a pressure control unit for controlling the pressure applied by the pressure adjustment unit, the flexible coil can fit the tissue under test in a relatively comfortable fashion, and hence the quality of magnetic resonance imaging can be improved.

In addition, the use of an airbag-style pressure adjustment unit based on the principle of gas pressure adjustment enables the pressure applied to the flexible coil to be gentle and uniform. Furthermore, because the amount of inflation is adjustable, it is possible to provide pressure that makes the tissue under test feel comfortable. At the same time, an elastic bandage-style pressure adjustment unit based on the principle of elastic adjustment can also achieve a similar effect.

In addition, because the adhesion position of the nylon clasp is adjustable, the use of the nylon clasp on the flexible coil for fixing makes the flexible coil suitable for tissues under test of various sizes and shapes.

Furthermore, when the flexible coil and tissue under test are put into the fixed support, by adjusting the amount of inflation of the airbag so that it fills the fixed support, the flexible coil and the tissue under test can be conveniently and stably fixed on the fixed support, making it easier to ensure the stability of the tissue under test when magnetic resonance imaging is subsequently performed.

### BRIEF DESCRIPTION OF THE DRAWINGS

Preferred aspects of the present disclosure are described in detail below with reference to the accompanying drawings, to give those skilled in the art a clearer understanding of the abovementioned and other features and advantages of the present disclosure. Drawings:

FIGS. 1A and 1B are exemplary structural drawings of a magnetic resonance coil assembly in aspects of the present disclosure.

FIGS. 2A and 2B are exemplary structural drawings of a flexible coil in an example of the present disclosure.

FIGS. 3A and 3B are schematic drawings of a flexible coil assembly being used in an example of the present disclosure.

FIGS. 4A and 4B are schematic drawings of a flexible coil assembly being used in another example of the present disclosure.

### Key to the Drawings

### Label Meaning

1 flexible coil

11 flexible coil body

12 flexible protective base layer

13 nylon clasp

2 pressure adjustment unit

3 pressure control unit

4 connection member

5 electronics box

6 first electric cable

7 second electric cable

8 electric cable plug

9 arm

10 fixed support

### DETAILED DESCRIPTION

The aspects of the present disclosure take into consideration the fact that at the present time, certain local coils are unable to achieve close contact with a patient's body; this is particularly true of coils for the four limbs, such as knee coils, hand/wrist coils, foot/ankle coils, etc. Consequently, the antenna unit is quite far away from the body tissue being scanned; moreover, the distance from the tissue to the antenna unit varies depending on the position of the tissue, and this will have an adverse effect on image quality. For this reason, in the aspects of the present disclosure, we consider providing a flexible coil assembly with an adjustable degree of fitting to body tissue. For example, first of all, a flexible coil capable of covering tissue under test in a wound fashion is provided. A pressure adjustment unit integrated with the flexible coil is then provided, for the purpose of applying uniform pressure to the entire flexible coil such that the flexible coil fits the covered tissue under test. To control the pressure applied to the flexible coil by the pressure adjustment unit, a pressure control unit is further provided, as well as a connection member connecting the pressure control unit to the pressure adjustment unit.

In particular implementation, the implementation of the pressure adjustment unit and pressure control unit can be based on the principle of gas pressure adjustment, or on the principle of elastic adjustment, or on another suitable principle. No restrictions are imposed here in this respect.

In order to clarify the object, technical solution and advantages of the present disclosure, the present disclosure is explained in further detail below by way of aspects.

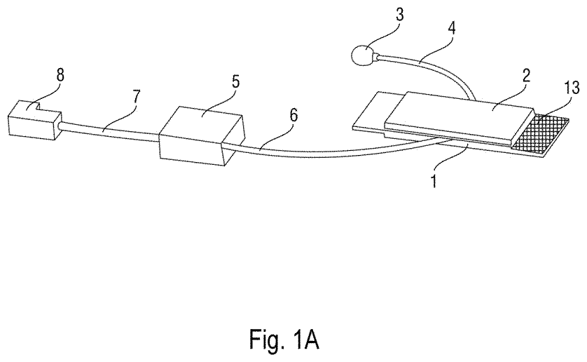

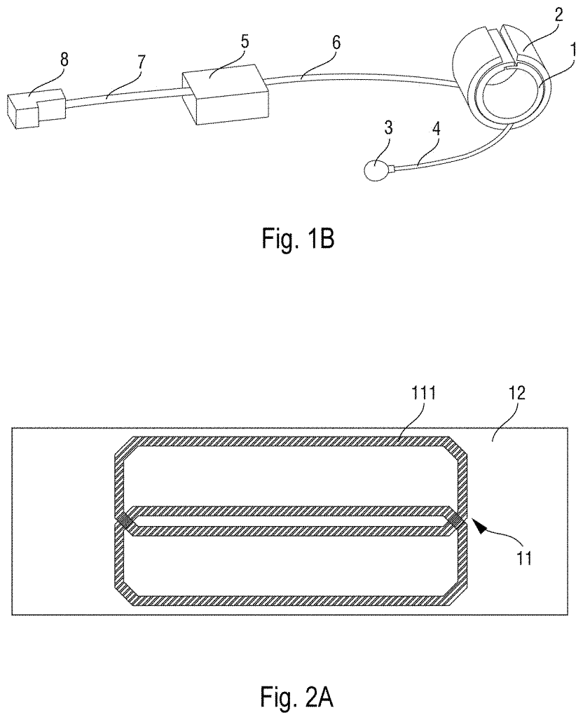

FIGS. 1A and 1B are exemplary structural drawings of a magnetic resonance coil assembly in an aspect of the present disclosure. As shown in FIGS. 1A and 1B, the magnetic resonance coil assembly comprises: a flexible coil 1, a pressure adjustment unit such as an airbag 2 in the figure, a pressure control unit such as a hollow latex ball 3 in the figure, a connection member such as an inflation tube 4 in the figure, an electronics box 5, a first electric cable 6, a second electric cable 7 and an electric cable plug 8.

The flexible coil 1 may be a corresponding local coil, such as a knee coil, hand/wrist coil, foot/ankle coil or shoulder coil, etc., being used to cover in a wound fashion the tissue under test, e.g. a knee, hand/wrist, foot/ankle or shoulder, etc.

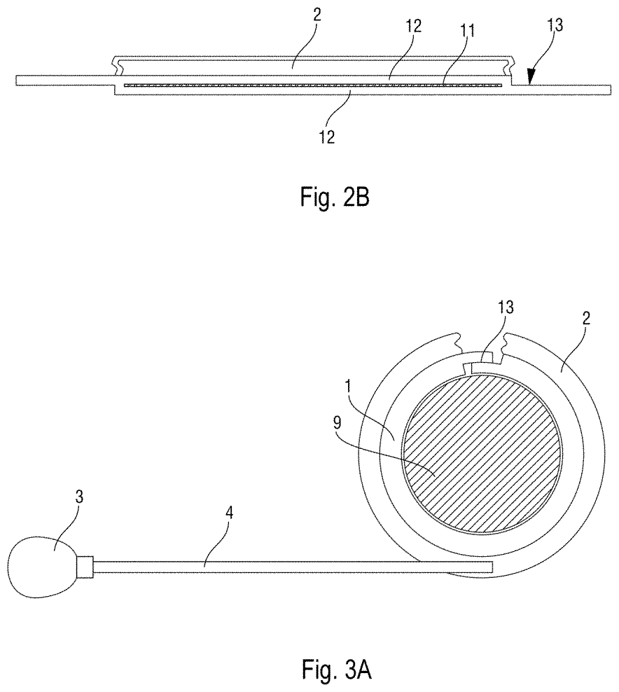

In particular implementation, the flexible coil 1 may be implemented in various ways; exemplary structural drawings of the flexible coil 1 in an example of the present disclosure are shown in FIGS. 2A and 2B. As shown in FIGS. 2A and 2B, the flexible coil 1 may comprise a flexible coil body 11, a flexible protective base layer 12 and a nylon clasp 13. The flexible coil body 11 comprises multiple rows of flexible antenna units 111 for receiving magnetic resonance signals, wherein decoupling of adjacent flexible antenna units can be achieved by overlapping. The flexible protective base layer 12 can be two layers arranged at two sides of the flexible coil body 11 respectively, being used to carry and protect the flexible coil body 11, and can be made of polyethylene foam. In particular implementation, the flexible coil body 11 and the flexible protective base layers 12 at the two sides can be pressed together by mold pressing. The nylon clasp 13 is arranged at two ends of the flexible protective base layer 12, in order to lock the position of the flexible coil 1 that covers the tissue under test in a wound fashion. The adhesion position of the nylon clasp is adjustable; thus, the use of the nylon clasp for fixing makes the flexible coil suitable for tissues under test of various sizes and shapes. In addition, in other aspects, the flexible coil 1 may also comprise some other associated components, as long as these do not affect the flexibility of the flexible coil 1.

The pressure adjustment unit is integrated with the flexible coil 1, for the purpose of applying uniform pressure to the entire flexible coil 1 such that the flexible coil 1 fits the covered tissue under test.

The pressure control unit is used to control the pressure applied to the flexible coil 1 by the pressure adjustment unit 2.

The connection member is used to connect the pressure control unit 3 to the pressure adjustment unit 2.

In particular implementation, the pressure adjustment unit, pressure control unit and connection member can have different specific structures based on different implementation principles. For example, FIG. 1 shows the case where implementation is based on gas pressure; as shown in FIG. 1, the pressure adjustment unit in this example is the airbag 2 fitting the entire magnetic resonance flexible coil 1, the pressure control unit is the hollow latex ball 3, and correspondingly, the connection member is the inflation tube 4. A gas inlet (not shown in the figure) and a one-way gas intake valve (not shown in the figure) may be provided on the hollow latex ball 3, and a gas release switch (not shown in the figure) may be provided on the inflation tube. By controlling the inflow and outflow of air, the hollow latex ball 3 and inflation tube 4 control the inflation of the airbag 2 so that it enters an inflated state or a shrunken state. In other aspects, the hollow latex ball 3 can also be replaced by an electronic controller with a gas pump and a pressure relief means; in this case, no gas release switch need be provided on the inflation tube 4. No restrictions are imposed here in this respect.

In addition, there may be an active connection between the inflation tube 4 and the airbag 2; thus, when the airbag 2 has been inflated, the inflation tube 4 and the hollow latex ball 3 or electronic controller can be removed, to facilitate magnetic resonance imaging. As another example, in other aspects, the pressure adjustment unit may also be an elastic element, such as an elastic bandage. Correspondingly, the pressure control unit may be a tension control element, and the connection member may be an intermediate pulling element.

The electronics box 5 is configured to accommodate a hard, large printed circuit board and associated electronic components, so as to avoid a reduction in the flexibility of the flexible coil 1 which would occur if the hard, large printed circuit board or electronic components were positioned on the flexible coil 1.

The first electric cable 6 is used for electrically connecting the flexible coil 1 to the electronics box 5.

The second electric cable 7 is used for electrically connecting the electronics box 5 to the electric cable plug 8.

The electric cable plug 8 is used for connecting the coil to an MRI system via the electric cables.

In addition, in other aspects, the electronics box 5, first electric cable 6, second electric cable 7 and electric cable plug 8 may also be replaced by other forms of implementation; no restrictions are imposed here in this respect.

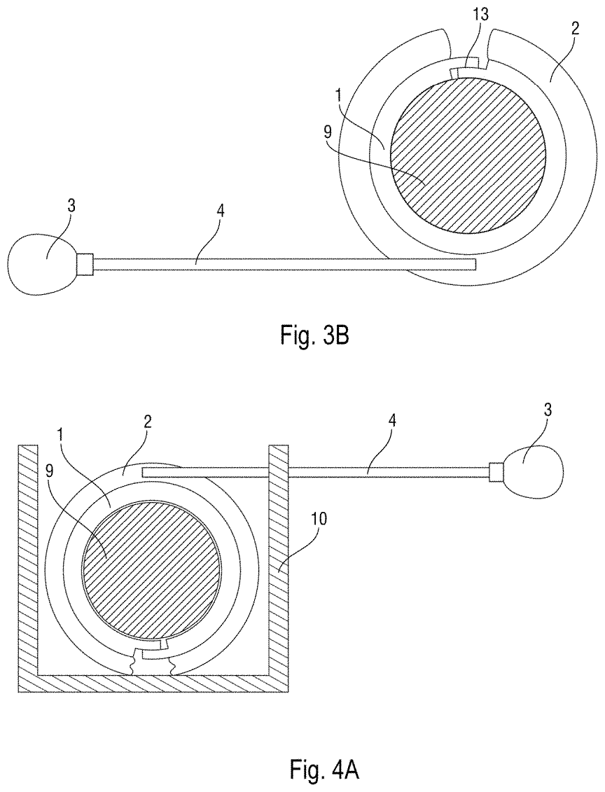

FIGS. 3A and 3B show schematic drawings of the flexible coil assembly being used in one example. As shown in FIG. 3A, the tissue under test is covered by the flexible coil 1 in a wound fashion; in the figure, an arm 9 is taken as an example. As can be seen, at this time, there is a certain gap between the flexible coil 1 and the arm 9, and by operating the pressure control unit, the pressure adjustment unit is controlled to apply uniform pressure to the entire flexible coil 1, so that the flexible coil 1 fits the covered tissue under test. Specifically, with implementation based on gas pressure in this aspect, the airbag 2 can be inflated by squeezing the hollow latex ball 3; while expanding under inflation, the airbag 2 applies uniform pressure to the entire flexible coil 1, so that the flexible coil 1 fits the covered tissue under test, thereby attaining the state shown in FIG. 3B.

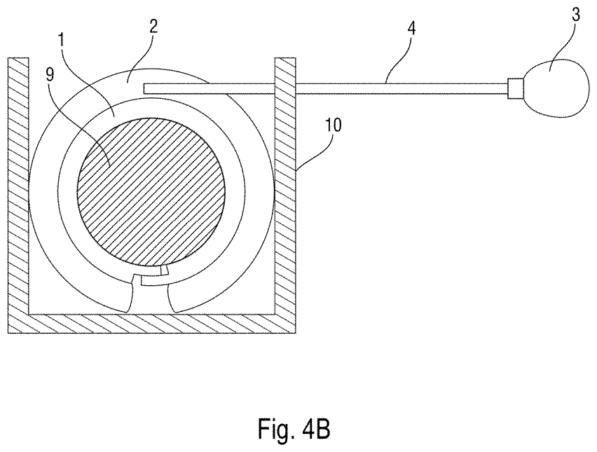

FIGS. 4A and 4B show schematic drawings of a flexible coil assembly based on gas pressure being used in another example. FIG. 4A shows the state before inflation; FIG. 4B shows the state after inflation. As shown in FIG. 4A, in the shrunken state, the flexible coil 1 and the tissue under test such as the arm 9 are put into a fixed support 10, e.g. the U-shaped fixed support in the figure, and the airbag 2 is inflated by squeezing the hollow latex ball 3 until the airbag 2 fills the fixed support, as shown in FIG. 4B. At this time, the flexible coil 1 and the tissue under test such as the arm 9 can be conveniently and stably fixed on the fixed support 10, making it easier to ensure the stability of the tissue under test when magnetic resonance imaging is subsequently performed. That is to say, the fixed support 10 is used to accommodate the flexible coil 1 and the tissue under test that is covered by the flexible coil in a wound fashion, and to fix and position the flexible coil 1 and the tissue under test when the airbag 2 expands under inflation to fill the fixed support 10.

Also proposed in the aspects of the present disclosure is an MRI apparatus, which may comprise the flexible coil assembly in any one of the aspects above.

It can be seen from the above analysis that because, in aspects of the present disclosure, the flexible coil is provided with a pressure adjustment unit integrated with the flexible coil and capable of applying uniform pressure to the entire flexible coil 1, and is also provided with a pressure control unit for controlling the pressure applied by the pressure adjustment unit, the flexible coil can fit the tissue under test in a relatively comfortable fashion, and hence the quality of magnetic resonance imaging can be improved.

In addition, the use of an airbag-style pressure adjustment unit based on the principle of gas pressure adjustment enables the pressure applied to the flexible coil to be gentle and uniform. Furthermore, because the amount of inflation is adjustable, it is possible to provide pressure that makes the tissue under test feel comfortable. At the same time, an elastic bandage-style pressure adjustment unit based on the principle of elastic adjustment can also achieve a similar effect.

In addition, because the adhesion position of the nylon clasp is adjustable, the use of the nylon clasp on the flexible coil for fixing makes the flexible coil suitable for tissues under test of various sizes and shapes.

Furthermore, when the flexible coil and tissue under test are put into the fixed support, by adjusting the amount of inflation of the airbag so that it fills the fixed support, the flexible coil and the tissue under test can be conveniently and stably fixed on the fixed support, making it easier to ensure the stability of the tissue under test when magnetic resonance imaging is subsequently performed.

The aspects above are merely preferred aspects of the present disclosure, which are not intended to limit it. Any amendments, equivalent substitutions or improvements etc. made within the spirit and principles of the present disclosure shall be included in the scope of protection thereof.

1. A magnetic resonance coil assembly, comprising:
a flexible coil (configured to cover a tissue under test in a wound fashion;
a pressure adjuster configured to be integrated with the flexible coil (to apply uniform pressure to the entire flexible coil such that the flexible coil fits the covered tissue under test;
a pressure controller configured to control the pressure applied to the flexible coil by the pressure adjuster; and
a connection configured to connect the pressure controller to the pressure adjuster.

2. The magnetic resonance coil assembly of claim 1, wherein:
the pressure adjuster is an airbag;
the connection member is an inflation tube; and
the pressure controller is a hollow latex ball, or an electronic controller with a gas pump and a pressure relief.

3. The magnetic resonance coil assembly of claim 2, further comprising:
a fixed support to accommodate the flexible coil and the tissue under test that is covered by the flexible coil in a wound fashion, and to fix and position the flexible coil and the tissue under test when the airbag expands under inflation to fill the fixed support.

4. The magnetic resonance coil assembly of claim 1, wherein:
the pressure adjuster is an elastic element;
the connection member is a pulling element; and
the pressure controller is a tension control element.

5. The magnetic resonance coil assembly of claim 1, wherein the flexible coil comprises:
a flexible coil body comprising multiple rows of flexible antenna units for receiving magnetic resonance signals;
a flexible protective base layer comprising two protective base layers arranged on two sides of the flexible coil body, and used to carry and protect the flexible coil body; and
a nylon clasp arranged at two ends of the flexible protective base layer, in order to lock the position of the flexible coil covering the tissue under test in a wound fashion.

6. The magnetic resonance coil assembly of claim 1, further comprising: an electronics box, a first electric cable, a second electric cable and a current plug, wherein:
the electronics box is constructed to accommodate a printed circuit board and electronic components;
the first electric cable is constructed to electrically connect the flexible coil to the electronics box; and
the second electric cable is constructed to electrically connect the electronics box to the electric cable plug.

7. An MRI system, comprising the magnetic resonance coil assembly of claim 1.

8. A method of using the magnetic resonance coil assembly of claim 1, comprising:
covering the tissue under test with the flexible coil in the magnetic resonance coil assembly in a wound fashion; and
controlling the pressure adjuster in the magnetic resonance coil assembly to apply uniform pressure to the entire flexible coil by operating the pressure controller in the magnetic resonance coil assembly, so that the flexible coil fits the covered tissue under test.

9. The method of use of claim 8, wherein the pressure adjuster is the airbag, the connection member is the inflation tube, and the pressure controller is the hollow latex ball; and
wherein the step of controlling the pressure adjuster in the magnetic resonance coil assembly to apply uniform pressure to the entire flexible coil by operating the pressure controller in the magnetic resonance coil assembly, so that the flexible coil fits the covered tissue under test, comprises: inflating the airbag by squeezing the hollow latex ball, the airbag applying uniform pressure to the entire flexible coil while expanding under inflation, so that the flexible coil fits the covered tissue under test.

10. The method of use of claim 9, further comprising:
putting the flexible coil and the tissue under test into a fixed support; and
inflating the airbag by squeezing the hollow latex ball, until the airbag fills the fixed support.
